SEQUENCE LISTING (12 sequences)

SEQ ID NO: 1 - protein, 30 aa, Artificial Sequence
  Primer
CCAGCCAAGATGTGGTGACCAAGACTGATT

SEQ ID NO: 2 - protein, 30 aa, Artificial Sequence
  Primer
CCAAGCAGCGGCCACCCAGGAGCACCTGCC

SEQ ID NO: 3 - protein, 30 aa, Artificial Sequence
  Primer
AGGTGGAACTGACAGTGTAGAGGGAATTCT

SEQ ID NO: 4 - protein, 29 aa, Artificial Sequence
  Primer
TGCACAAGCTGCAGTTGACGTAGCTCGAG

SEQ ID NO: 5 - protein, 30 aa, Artificial Sequence
  Primer
CATGGGTCATCTCGGCGCCAGAGCCGCTCT

SEQ ID NO: 6 - protein, 30 aa, Artificial Sequence
  Primer
AGCCAGCTGCCCCGTATGACCGCGCCGGGC

SEQ ID NO: 7 - protein, 30 aa, Artificial Sequence
  Primer
GGAGTATCACCGAGGAGGTGTCGGAGTACT

SEQ ID NO: 8 - protein, 14 aa, Artificial Sequence
  Primer
GCCCGGCGCGGTCA

SEQ ID NO: 9 - protein, 14 aa, Artificial Sequence
  Primer
ACGGGGCAGCTGGC

SEQ ID NO: 10 - protein, 14 aa, Artificial Sequence
  Primer
CGAGAGGACCCAGG

SEQ ID NO: 11 - protein, 15 aa, Artificial Sequence
  Primer
ATGACCGCGCCGGGC

SEQ ID NO: 12 - protein, 21 aa, Artificial Sequence
  Primer
CACTGGCAGTTCCACCTGTCT

(19) United States
(12) Patent Application Publication
(10) Pub. No.: US 2009/0317403 A1
(43) Pub. Date: Dec. 24, 2009

(54) Use of CSF-1 Inhibitors

(76) Inventors: Seyedhossein Aharinejad, Vienna (AT); Reinhold Hofbauer, Vienna (AT)

Correspondence Address: BUCHANAN, INGERSOLL & ROONEY PC, POST OFFICE BOX 1404, ALEXANDRIA, VA, 22313-1404, US

(21) Appl. No.: 12/465,490

(22) Filed: May 13, 2009

Related U.S. Application Data
(63) Continuation of application No. 10/111,711, filed on Jun. 7, 2002.

(30) Foreign Application Priority Data
Oct. 28, 1999 (AT) A1813/99

(51) Int. Cl. A61K 39/395; A61K 48/00; A61K 35/00; A61P 35/00

(52) U.S. Cl. 424/158.1; 514/44 A; 424/93.21

(57) ABSTRACT

Disclosed is the use of inhibitors of CSF-1 activity for preparing a medicament for the treatment of tumor diseases.

This application is a continuation of U.S. patent application Ser. No. 10/111,711, filed on Jun. 7, 2002, which is the U.S. National Stage of International Application No. PCT/AT00/00281, filed on Oct. 25, 2000, which claims priority to Austrian Application No. A1813/99, filed on Oct. 28, 2009. The entire teachings of the above applications are incorporated herein by reference.

The invention relates to the use of inhibitors of CSF-1 activity.

The colony stimulating factor 1 (CSF-1) is a cytokine capable of primarily forming macrophage colonies. Native CSF-1 is a glycosylated dimer, various forms of this molecule having various lengths and various molecular weights being present in humans. It is, e.g., known that the two main forms of CSF-1 having 224 and 522 amino acids, respectively, are formed by alternative splicing. Furthermore, it is known that the minimum length of this factor is approximately 150 amino acids. Moreover, CSF-1 may also occur in various glycosylation patterns which are specific depending on the physiological state, or tissue-specific.

CSF-1 has been used to overcome the immune suppression in patients which, e.g., has been caused by

CSF-1 has been used to overcome the immune suppression in patients which, e.g., has been caused by chemotherapy. Further applications related to the treatment or prevention of bacterial, viral or fungus-caused infections, the stimulation of white blood cells and the assistance in wound healing.

Moreover, CSF-1 has also been used for the treatment of tumor diseases (U.S. Pat. No. 5,725,850), and this not only to support immune suppressed tumor patients, but also for the direct killing of tumor cells. In this case it has been found that primarily sarcoma tumor cells can be killed by administering CSF-1 (U.S. Pat. No. 5,104,650).

However, the anti-tumor effect of CSF-1 is not undisputed in the prior art; thus Anderson et al. (Gynecol. Oncol. 74(2) (1999), 202-207) have reported that neither CSF-1, nor its receptor, play a role in the pathogenesis of uterine sarcomas. On the other hand, it is known that both CSF-1 and also its receptor for endometric adenocarcinomas correlate with the tumor progression. Finally, in CSF-1-deficient and macrophage-deficient mice, a reduced tumor growth could be found with one special tumor (Lewis lung carcinoma), yet despite the reduced tumor growth, the CSF-1 deficient mice died more quickly then the tumor-carrying control mice (Nowicki et al., Int. J. Cancer 65 (1996), 112-119). It has been assumed that the reduced life expectancy was also a consequence of the massive necrosis formation in the CSF-1 deficient mice.

Accordingly, the role of CSF-1 as an anti-tumor agent has, indeed, remained disputed, yet a negative effect of CSF-1 on the treatment of tumors so far has not been discussed in the prior art or considered possible.

The present invention has as its object to provide an agent for treating tumor patients, in particular with the inclusion of the role which CSF-1 plays in tumors.

According to the invention, this object is achieved by the use of CSF-1-activity-inhibiting compounds for preparing an agent for the treatment of tumor diseases. In the course of the present invention it has surprisingly been found that—contrary to the effects hitherto suggested in the prior art —CSF-1 itself does not have any anti-tumor effect, but that the tumor growth can be retarded or prevented by administering compounds which inhibit CSF-1 or its receptor, and that this leads to an increased survival rate. It has, indeed, been known in the prior art that CSF-1 correlates in some tumors with the progression of tumor growth, yet so far it has been assumed that this content of CSF-1 and CSF-1 receptor would not have any influence on tumor growth; on the contrary, in the prior art it has been assumed that an increased CSF-1 production has led to a retrogression of tumors. Thus, U.S. Pat. No. 5,725,850 does disclose that increased CSF-1 concentrations can be employed to stimulate macrophages which kill mouse sarcoma TU5 cells, yet it is also mentioned that actually this activity is really effective only if CSF-1 is used in combination with interleukin-2, IFN-α, IFN-β or IFN-γ. Thus, possibly this sarcoma-killing effect reported in the prior art could have been due to the additional lymphokines administered with CSF-1.

In contrast, it has been recognized within the scope of the present invention that the administration of CSF-1 inhibiting substances or of CSF-1 receptor-inhibiting substances in fact has an anti-tumor effect. This is in contrast to the teaching so far spread in the prior art.

The only effect which, so far, with the knowledge of the present invention, points towards a negative effect of CSF-1 in connection with tumor diseases, hitherto has been a hindered tumor growth in CSF-1-deficient, macrophage-deficient mice. In this connection, the role of CSF-1-dependent macrophages in the formation of tumorstroma has been pointed out (cf. Nowicki et al.), by concluding that the LLC tumor growth in CSF-1-deficient mice is not facilitated by the absence of CSF-1-dependent macrophages (as actually could have been expected on the basis of the anti-tumor effect of CSF-1 itself hitherto described in the prior art). There, also the significant anti-tumor effects which could be shown in the in vivo-treatment of mice with CSF-1 have been pointed out. Although it has been shown in CSF-1-deficient mice in which an LLC tumor was implanted that the tumor growth was not increased relative to normal mice, but that in fact, the deficient mice had little stroma tissue. The LLC tumors in these animals were substantially more necrotic; this was also seen as the cause of the reduced growth. In any event, the CSF-1-deficient mice died earlier than the tumor-suffering control mice. Nowicki et al. first of all stated that the LLC tumor is not a representative tumor to demonstrate the role of CSF-1 in natural anti-tumor immunity. In the Nowicki et al.—model, this tumor has merely been used because it grew reproducibly both in control mice and in CSF-1 mice.

Likewise, it has been stated by Nowicki et al. that the data obtained with CSF-1-deficient mice do not contradict the hypothesis that CSF-1-dependent macrophages play an important role in the induced anti-tumor response, particularly if a stimulus with exogenous CSF-1 takes place, as has been reported in the prior art.

In fact, however, within the scope of the present invention it has been found that it is not the administration of CSF-1 itself which triggers an anti-tumor response or can be used for the treatment of tumor diseases, respectively, but that an efficient tumor treatment can be achieved by inhibiting CSF-1 activity.

Accordingly, the present invention relates to the use of inhibitors of CSF-1 activity for preparing a medicament for the treatment of tumor diseases. The inventive agent for treating tumor diseases which comprises inhibitors of CSF-1 activity, thus is in contrast to the prevailing teaching in which rather CSF-1 itself has been attributed an anti-tumor effect, or at least a neutral role of CSF-1 has been assumed in most tumor diseases.

With the present invention, in a method of treating tumor diseases, an efficient dose of inhibitors of CSF-1 activity is administered to a tumor patient.

The manner in which the CSF-1 activity is inhibited is not critical. In the prior art, a whole number of CSF-1 activity-inhibiting substances have been described.

The two essential approaches for the inhibition of CSF-1 activity are the suppression of the CSF-1 activity itself, and the suppression of the activity of the CSF-1 receptors (cf. U.S. Pat. No. 5,405,772).

According to the invention, neutralizing antibodies against CSF-1 or its receptor are preferred as the inhibitors of CSF-1 activity. Such neutralizing antibodies (described e.g. in Weir et al., J. Bone and Mineral Research 11 (1996), 1474-1481) bind CSF-1 or the CSF-1 receptor such that a CSF-1 activity is inhibited or is not made effective, respectively.

Alternatively, CSF-1 activity can be inhibited with the assistance of antisense technology, in which short sequences of single-stranded nucleic acids are used to prevent the expression of CSF-1 or of its receptor or of another part of the signal transducing mechanism of CSF-1 activity. The person skilled in the art is familiar with the antisense technology (e.g. in “Antisense Technology—A Practical Approach”, Lichtenstein and Nellen (eds.), IRL Press, Oxford University Press 1997, and “Oligonucleotides as Therapeutic Agents”, Ciba Foundation Symposium 209, John Wiley & Sons 1997; included herein by reference) and can easily adapt it for CSF-1 or the CSF-1 receptor with any suitable sequence.

Sequences which as a whole or as an effective fragment thereof are to be considered for the antisense-treatment are i.a. described in U.S. Pat. Nos. 4,847,201, 5,792,450, 5,681,719, 5,861,150, 5,104,650 and 5,725,850, included herein by expressly referring thereto.

Furthermore, also synthetic inhibitors of CSF-1 activity can be employed within the scope of the present invention.

The inventive inhibition of the CSF-1 activity is particularly suitable for inhibiting or retarding the growth of solid tumors.

The method according to the invention has proved particularly efficient for the treatment of solid tumors selected from the group of germinal tumors, epithelial tumors and adenocarcinomas. Malignant diseases of the hematopoietic system (e.g. leukemias) are not treatable.

Besides the afore-mentioned preferred inhibitions of the CSF-1 activity by neutralizing the antibodies or by using antisense technology, or by using chemical inhibitors and competitors of CSF-1 or its receptor, according to the invention cells or cells of the solid tumor can be genetically altered such that they counteract the growth and the development of the solid tumor. By methods of gene therapy, the activity of CSF-1 or the activity of the CSF-1 receptor is inhibited by the induced expression of genetically altered CSF-1 or its receptor or a mutant thereof, in particular by deletion of at least parts of the gene coding for CSF-1 or its receptor.

Particularly with this cellular inhibitor for which, according to the invention, all suitable cell types can be used (except for cells of the germ line), the medicament to be prepared according to the invention is formulated for intra-tumoral administration so that it can be employed directly at the site of the tumor. This is also a preferred variant of administration for the remaining inhibitors.

The medicament according to the invention may, however, also be administered in other ways, in particular topically, intravenously, intra-arterially, subcutaneously, intraperitoneally, intrapleurally, intrathecally or in combination with cationic lipids.

As previously mentioned, a particularly preferred variant of the present invention consists in the use of the antisense method, i.e. in a method in which certain regions of an mRNA that codes for CSF-1 or its receptor, are present in inverse direction, are used. Accordingly, the inventive inhibition of the CSF-1 activity can also be caused by means of gene-therapeutically expressible CSF-1 antisense constructs.

These CSF-1 antisense construct may, e.g., be prepared by carrying out the following steps:
a) amplification of CSF-1 DNA by means of PCR
b) sub-cloning of the PCR product of CSF-1 in an antisense orientation
c) insertion of the step b) E. coli DNA and
d) isolation of the E. coli-amplified CSF-1 antisense construct.

During amplification of CSF-1-DNA by means of PCR, either slight amounts of a CSF-1-cDNA or of a cDNA-library are amplified by the addition of appropriate Taq-DNA polymerase. The amplification product, i.e. the PCR product of CSF-1, subsequently is subcloned in its antisense orientation into a vector, whereupon the recovered recombinant DNA, i.e. the CSF-1 antisense sequence which has been cloned into the vector, is introduced in E. coli by transformation and amplified, whereupon the E. coli-amplified CSF-1 antisense construct, i.e. the plasmid, is isolated from the bacterial cells by standard methods and supplied for further use. For the isolation, e.g. the per se known method of alkaline lysis for plasmid isolation may be employed. A subsequent sequencing of the amplified and cloned CSF-1 antisense constructs may be carried out. This method is characterized by a particular simplicity and precision, and with this inventive method it is possible to quickly and reliably obtain high yields of specifically active CSF-1 antisense constructs. The details of the method may be described in that the following steps are carried out:
a) amplification of CSF-1-DNA by means of PCR
b) sub-cloning of the PCR product of CSF-1 in an antisense orientation
c) amplification of the CSF-1 antisense-cDNA constructs obtained in step b) and
d) integration in recombinant viral transfer vectors
e) amplification of the constructs obtained in step c) and co-transfection of the latter together with adenovirus-DNA in cell culture cells
f) recombination of the CSF-1 antisense-cDNA-constructs with adenovirus-DNA and
g) amplification of the recombinants in cell culture cells,
h) preparation and purification of the recombinant adeno-viral CSF-1 antisense constructs
i) and their use in mammalian organisms (gene therapy of cell culture tumor cells), test animals (mouse, rat), use in tumor patients
j) selection of CSF-1 primary sequence regions suitable for oligonucleotide antisense inhibition
k) preparation and modification of nuclease-resistant CSF-1 antisense oligonucleotides
l) use of the latter in mammalian organisms (gene therapy of cell culture tumor cells), test animals (mouse, rat), use in tumor patients).

The amplification of total-CSF-1 (this method can, of course, be used 1:1 on the CSF-1 receptor) or also of parts thereof may preferably be carried out with 3′-primers or 5′-primers, respectively, the primer length in particular being 15 to 30 nucleotides, and for obtaining a particularly reliable and precisely targeted, in particular specific amplification, preferably the following 31 primers
ccagccaaga tgtggtgacc aagactgatt (Nucleotides No. 641-670) (SEQ ID NO: 1)
ccaagcagcg gccacccagg agcacctgcc (Nucleotides No. 851-880) (SEQ ID NO: 2)
aggtggaact gacagtgtag agggaattct (Nucleotides No. 1751-1780) (SEQ ID NO: 3)
tgcacaagct gcagttgacg tagctcgag (Nucleotides No. 3911-3939) (SEQ ID NO: 4)
   and 5′-primers, respectively,
catgggtcat ctcggcgcca gagccgctct (Nucleotides No. 1-30) (SEQ ID NO: 5)
agccagctgc cccgtatgac cgcgccgggc (Nucleotides No. 91-120) (SEQ ID NO: 6)
ggagtatcac cgaggaggtg tcggagtact (Nucleotides No. 191-220) (SEQ ID NO: 7) may be used.

To attain a particularly exact and specific amplification, the method according to the invention preferably is carried out such that the amplification of CSF-1 DNA is carried out with 20 to 40 cycles, in particular 25 to 35 cycles, for denaturing, annealing and extension in a PCR machine, a programmable PCR machine being particularly used for reasons of exactness of the course of the method. According to the invention, denaturing is carried out at 85° C. to 100° C. for 20 s to 4 min, in particular at 93° C. to 98° C. for 30 s to 2 min, whereby a complete, nearly 100% denaturing of the protein sequence is ensured. According to the invention, annealing is preferably carried out at 30° C. to 70° C. for 30 s to 4 min, in particular at 37° C. to 65° C. for 1 min to 2 min, wherein it can be ensured according to this method course that annealing will be carried out as completely as possible, wherein due to the wide temperature interval in which this method may be carried out, in particular also a method course suitable from the point of energy can be achieved, since after denaturing, the temperature for annealing need not necessarily be lowered to approximately the body temperature, as it is the case in many known methods. Finally, extension preferably occurs at 65° C. to 80° C. for 30 s to 6 min, in particular at 72° C. to 74° C. for 1 min to 4 min, wherein it results particularly from the entire method course in the PCR machine that the duration of the method can be kept relatively short despite the plurality of steps for obtaining a complete and specifically amplified total-CSF-1 or parts thereof.

To further simplify, in particular complete the method course, according to the invention the method preferably is carried out in such a way prior to the cycles for denaturing, annealing and extension and thereafter, respectively, that at the beginning of amplification, an additional denaturing step at approximately 95° C. is carried out for approximately 2 min, and at the end of amplification, a final extension at 72° C. to 74° C. is carried out for approximately 5 to 10 min. By this additional denaturing at the beginning of the reaction, a large percentage of the proteins is already denatured before the method cycles are carried out, which leads to a more complete turnover particularly in the first method cycles. Finally, it has been shown that by using a final extension the product yield could be further increased.

For sub-cloning the cDNA synthesized as a PCR product of CSF-1, it is preferably proceeded such that the cDNA synthesized as a PCR product of CSF-1 is subcloned into a plasmid vector, in particular pCRII, and integrated in the MCS of the pCRII vector by incubating for 1 to 24 h at 4° C. to 25° C. In doing so, at first sub-cloning into a plasmid vector is effected, the known vector pCRII preferably having proven to be suitable which may, e.g., be bought from InVitrogen. Integration of the cDNA into the MCS (i.e. the multiple cloning site) of the vector pCRII is effected by mild incubation according to the various known incubation methods, wherein it has been shown that a molar ratio of insert to vector of 1:3 results in a particularly reliable and complete ligation. When integrating the cDNA into the vector, the EcoRI recognition sequence of the MCS may, e.g., be used as the cleavage site, whereby a further improvement of the method of the invention can be obtained.

Finally, it has been found that a particularly efficient and reliable insertion of the DNA in E. coli can be obtained by preferably carrying out the insertion in E. coli by bacterial transformation by means of heat shock, by the shock-type heating of an ice-cooled mixture of E. coli cells and of DNA to be transformed, to approximately 40° C. to 44° C., in particular 42° C., and a subsequent rapid cooling in an ice bath as well as a subsequent incubation and culturing.

Another method also preferred according to the invention consists in that the insertion of the DNA in E. coli is effected by transformation of E. coli with plasmid DNA by electroporation, in particular at 25 μF, 2.5 kV and a resistance of 200 ohm and subsequent regeneration, incubation and culturing of the cell colony.

Both insertion procedures in E. coli are characterized by high yields when growing the colonies, and in this manner a sufficient amount of the inventive construct for a further use in carcinoma therapy can be obtained with a simple transformation method. A further advantage of the method according to the invention consists in that the construct is obtainable in high purity and with high selectivity so that a further purification after isolation of the construct is not necessary, whereby both the duration of the method as well as the costs of the method can clearly be lowered.

Besides the possibility of amplifying CSF-1 by means of PCR from an already existing cDNA library and to isolate it, preferably the CSF-1-DNA to be amplified by means of PCR can be prepared by isolation of whole-RNA from CSF-1 expressing cells, in particular from fibroblasts, or of mRNA, followed by a cDNA synthesis by reverse transcription with PCR. Such cloning methods are generally known in the art and had to be appropriately adapted and perfected so as to obtain the special CSF-1-DNA to be amplified by means of PCR. In doing so it has been shown that the whole-RNA from CSF-expressing cells, in particular from fibroblasts, can be obtained in a particularly preferred way by using the guanidinothiocyanate method for RNA extraction, wherein, for isolating the alternatively used messenger RNA, the oligo-dT-cellulose chromatography can be employed, which is a very specific reaction course in which very high yields of product can be obtained. The reverse transcription by means of PCR required after isolation of the whole-RNA or of the messenger-RNA may be carried out in a similar manner as described in the methods according to the invention, it having been shown with this method that the number of cycles on the PCR machine should be slightly increased so as to obtain complete, or selective products, respectively. Analogous considerations hold also for the final extension which suitably should be carried out for at least 10 min. However, with the isolation of whole-RNA or mRNA and subsequent cDNA synthesis proposed according to the invention, as compared to the method in which an mRNA library is used, an even more specific and purer product can be attained, this product being obtainable with merely slightly increased time consumption and increased costs.

To obtain a further improvement of the method course and, in particular, an even higher product specificity or purity, respectively, a purification via gel filtration may be carried out prior to ligation with adapters, whereby the starting product is purified from smaller fragments not required for the method course according to the invention. Moreover, the cloning efficiency will be increased by this method course, by phosphorylating the DNA and purifying the recovered cDNA by means of standard DNA purifying protocols or by using an affinity chromatography. A further increase of the yield and, in particular, an improvement in the purity may be obtained by an additional extraction with a TE buffer.

According to a further object, the invention aims at a method in which gene-therapeutically expressible CSF-1 antisense constructs are prepared, this object being achieved in that gene-therapeutically expressible CSF-1 antisense constructs are prepared by forming recombinant, infectious adenoviruses by excision of the CSF-1-cDNA from the plasmid vector and subsequent cloning in an antisense orientation into an adenoviral transfer vector. In doing so, the CSF-1-cDNA is cleaved from the plasmid vector, in particular pCRII, with restriction enzymes, and subsequently cloned in an antisense orientation into a transfer vector which in turn has been cleaved by restriction enzymes, whereupon E. coli is transformed in a manner known per se and subsequently a screening for recombinant plasmids is carried out. In this manner, the recombinant transfer vector which comprises the integrated CSF-1-cDNA in antisense orientation can be obtained in high yield. Subsequently, the recombinant transfer vector is inserted into adenoviral DNA so as to obtain an adenoviral transfer vector. In doing so, according to the invention it is preferably proceeded such that the infectious, recombinant adenoviruses are formed by homologous recombination between a transfer vector comprising an integrated CSF-1-cDNA, and an adenoviral genomic plasmid, in particular Ad5. By the fact that recombinant, adenoviral vectors are obtained by a homologous recombination between the transfer vector and the adenoviral, genomic plasmid, occurring in the present instance in the human tumor cell line 293, it is possible to obtain a product which comprises CSF-1 in antisense orientation, on the one hand, and which comprises a replication-defective virus, on the other hand, which is capable of propagating only in cells which provide the defective sites, such as, e.g., E1A- and E1B-genes, in trans-position, whereby a selective propagation of the viruses can be ensured. By this selective propagation of the replication-defective viruses a likewise selective use of the same is possible.

The recombinant Ad5 viruses used according to the invention are helper-independent viruses which can be propagated in the human cell line 293 preferably utilized according to the invention.

According to the invention, CSF-1-phosphorothioate-antisense oligonucleotides (5-propinyl analogues), CSF-1-methylphosphonate-antisense oligonucleotides, CSF-1-2′-O-methyl-antisense oligonucleotides or terminally modified CSF-1 antisense oligonucleotides or the corresponding antisense oligonucleotides for the CSF-1 receptor may also be used as the oligonucleotides. Such oligonucleotides are known in the prior art for the most varying growth factors and are prepared according to standard methods.

In the “antisense inhibiting technique” based on gene-specific oligodeoxynucleotides, a modification of the single-stranded, synthetic DNA molecule is necessary so as to increase its nuclease resistance. Phosphorothioate-modified oligonucleotides have a higher stability as compared to the non-modified oligonucleotides, a substitution of an O atom by S occurring at the phosphodiester bridge. In this manner, e.g., a longer activity can be obtained with lower amounts applied. Oligonucleotides modified in this manner have a higher resistance to an intra-cellular nuclease degradation and can be utilized according to the invention as antisense molecules to inhibit gene expression and as chemotherapeutic agents. Attention must be paid to the fact that, of course, also the oligonucleotides in therapeutical use may only be used in purified form so that shorter or faulty adducts or synthesis by-products will have been separated prior to use. According to the invention, both completely modified oligonucleotides and also merely partially modified, phosphorothioate-bridges-carrying oligonucleotides may be used, wherein, as mentioned before, the mode of action and the activity of the oligonucleotides differ slightly, with the terminally modified CSF-1 antisense oligonucleotides, e.g. having an increased affinity between the target sequence and the antisense oligonucleotide as well as an improved uptake into the cell, an increased resistance to a nuclease degradation and a better detectability. In principle, however, it must be stated that all the oligonucleotides in the carcinoma therapy can be employed analogously to the CSF-1 antisense constructs, the application according to the invention preferably being topically, intravenously, intra-arterially, subcutaneously, intra-peritoneally or in combination with cationic lipids.

The gene-therapeutically expressible CSF-1 antisense constructs also prepared and usable according to the invention are preferably administered intratumorally, since by the intra-tumoral administration it can be ensured that the replication-defective virus will be used for infection of the tumor cells of the body at the site required therefor. In principle, theoretically also the gene-therapeutically expressible CSF-1 antisense construct could be administered in conventional ways, such as topically, intravenously, intra-arterially, subcutaneously, etc., yet in this case the effectiveness seems clearly restricted.

By the preparation and use of CSF-1 antisense constructs, CSF-1 antisense oligonucleotides as well as gene-therapeutically expressible CSF-1 antisense constructs, thus the preparation and use of biological substances become possible which clearly inhibit the growth, and the multiplication, respectively, of carcinoma cells, thereby enabling a selective and targeted carcinoma therapy with the constructs prepared according to the invention.

According to a particularly preferred use, it is proceeded according to the invention such that as the CSF-1 sequences of nucleotide 1-180 (derived from the human CSF-1 gene sequence, EMBL acc. no. M37435, LOCUS: HUMCSDF1), in particular the following 14-mers ON-1CSFlas: 5-GCCCGGCGCGGTCA-3 ((SEQ ID NO: 8) 14-mer homologous to the first 14 nt following the start codon (ATG) (nucleotides 120-106)ON-2CSFlas: 5-ACGGGGCAGCTGGC-3 ((SEQ ID NO: 9) 14-mer homologous to the 14 nt in front of the start codon (ATG) nucleotides 105-91)ON-3CSFlas: 5-CGAGAGGACCCAGG-3 ((SEQ ID NO: 10) 14-mer homologous to the 14 nt following the transcription start of the mRNA (nucleotides 14-1) are used.

The invention will be explained in more detail by way of the following examples to which, of course, it shall not be restricted.

### EXAMPLE 1

### Preparation of the CSF-1-cDNA Constructs

To isolate whole-RNA from CSF-1 expressing cells (L929 fibroblasts) which are to be used as starting material, the guanidino-thiocyanate method is used for RNA extraction. It is proceeded as follows:
removing the medium from the L929 fibroblasts, adding 1 ml of denaturing solution and cell lysis by pipetting
transferring the homogenate in 5 ml tubes and adding 0.1 ml 2 M sodium acetate (pH 4), mixing, subsequently adding 1 ml of water-saturated phenol, mixing, adding 0.2 ml of chloroform/isoamyl alcohol (49:1), mixing and incubating the suspension at 0-4° C. for 15 min
centrifuging for 20 min at 4° C. and 10,000 g, transferring the aqueous phase to a new tube
precipitating the RNA by adding 1 volume of 100% isopropanol, cooling samples for 30 min to −20° C., then centrifuging at 4° C. for 10 min and 10,000 g, discarding supernatant solution
dissolving the above-formed RNA-pellet in 0.3 ml of denaturing solution
precipitating RNA with 0.3 ml of 100% isopropanol for 30 min at −20° C., then for 10 min at 4° C., and centrifuging at 10,000 g and discarding the supernatant solution
resuspending the RNA pellet in 75% ethanol, vigorous stirring and incubating for 10-15 min at room temperature
centrifuging for 5 min at 10,000 g, discarding supernatant solution and drying RNA pellet in vacuum for 5-15 min
dissolving the RNA pellet in 200 μl of DEPc treated water, quantifying RNA by means of UV spectrophotometry at 260 nm.

Amplification of the CSF-1 RNA by means of RT-PCR (reverse transcriptase PCR).

Put 1 μg of the recovered CSF-1 RNA into a microcentrifuge tube, and incubate for 10 min at 70° C., centrifuge shortly, then put on ice.

Preparation of a 20 μl reaction by adding the following reagents to CSF-1 RNA:

| | | |
|---|---|---|
| MgCl₂, 25 mM | 4 | μl |
| Reverse transcription buffer, 10x | 2 | μl |
| dNTP mixture, 10 mM | 2 | μl |
| Rnasin ribonuclease inhibitor | 0.5 | μl |
| AMV reverse transcriptase | 15 | units |
| Oligo(dT) primer | 0.5 | μg |
| CSF-1 RNA | 1 | μg |
| Nuclease-free water to a total volume of | 20 | μl |

Subsequently, the reaction is incubated at 42° C. for 15 min, and then it is heated at 99° C. for 5 min and again incubated at 0-5° C. for 5 min. For amplification, the solution is diluted as follows: The first strand cDNA synthesis reaction is diluted with nuclease-free water to 100 μl, and subsequently the 50 μl PCR amplification reaction mixture is prepared by combining the following reagents (template-specific upstream and downstream primers must be added here, i.e. CSF-1 specific primers):

for 5′-primer:

(SEQ ID NO: 11)

CSF1 sense 5′-atgaccgcgccgggc
(Nucleotides No. 106-120)
for 3′-primer:

(SEQ ID NO: 12)

CSF1 antisense 5′-cactggcagttccacctgtct
(Nucleotides No. 1767-1747)

The following PCR reaction mixture was used: H₂O

| | Volume | | Final Concentration | |
|---|---|---|---|---|
| MgCl₂, 25 mM | 3 | μl | 1.5 | mM |
| 10X PCR buffer | 5 | μl | 1x | |
| dNTP, 10 mM | 1 | μl | 200 μM of each | |
| upstream primer | 5-50 | pM | 0.1-1 | μM |
| downstream primer | 5-50 | pM | 0.1-1 | μM |

| | | | |
|---|---|---|---|
| Taq DNA polymerase, | 0.25 | μl | 1.25 Units/50 μl |
| 5 u/μl | | | |
| first strand cDNA | 10 | μl | |
| reac. | | | |
| nuclease-free H₂O to | 50 | μl | |
| a vol. of | | | |

In this instance, the addition of Taq polymerase was last.

The PCR machine was programmed with the times and temperatures for denaturing, annealing and extension as follows:

| | | |
|---|---|---|
| Denaturing at reaction start: | 95° C. for 5 min 1 cycle | |
| Denaturing: | ca. 95° C. for 1:00 min. | |
| Annealing: | 65° C. for 1:00 min. | 35 cycles |
| Extension: | 72° C. for 2:00 min. | |

| | |
|---|---|
| Final extension: | 72° C. for 5 min, after the last cycle. |

The mixture is kept at 4° C. until the PCR machine is switched off and the samples are removed. To each PCR reaction, 100 μl of chloroform are added, it is stirred, centrifuged for 2.00 min, and the upper phase is saved for further processing. For a size determination of the product, 10 μl of the PCR product are applied with DNA size markers on an agarose gel.

Subsequently, the PCR product is purified as follows:
Adding 250 μl of buffer PB to 50 μl of the PCR reaction.
A QIAQUICK® spin column is put into a 5 ml centrifuge tube.
The sample is loaded on the column and centrifuged at 3000 g for 1 min.
Washing: Adding 0.75 ml of buffer PE and centrifuging for 1 min.
Transferring the QIAQUICK® column to a microcentrifuge tube. Centrifuging for 1 min at 10,000 g.
Put the QIAQUICK® column into a 1.5 ml reaction vessel.
Eluting the DNA by adding 50 μl 10 mM Tris-Cl, pH 8.5, and centrifuging for 1 min at max. speed in a microcentrifuge.
Collected eluate: ca. 48 μl. The DNA concentration is determined by means of UV spectrophotometry at 260 nm.

For further reaction, suitably an EcoRI adapter ligation is carried out as follows:

| | |
|---|---|
| T4 DNA ligase 10X buffer | 3 μl |
| acetylated BSA, 1 mg/ml | 3 μl |
| cDNA (50 ng/μl) | 5 μl |
| adapters (20-fold molar excess: | 1 μl |
| 10 pmol adapter) | |
| T4 DNA ligase (Weiss units) | 2.5 μl |
| fill up with nuclease-free water to | 30 μl |

The formed solution is incubated over night at 15° C., the enzyme is inactivated by heating the reaction mixture at 70° C. for 10 min, and finally the reaction is cooled on ice.

For carrying out the reaction without any problems, the insert DNA is phosphorylated as follows:

| | |
|---|---|
| ligation mixture | 30 μl |
| T4 PNK 10X buffer | 4 μl |
| ATP, 0.1 mM (1:100 dilution | 2 μl |
| of a 10 mM stock solution in water) | |
| T4 PNK (10 u/μl) | 1 μl |
| nuclease-free water | 3 μl |
| total volume | 40 μl |

The solution is incubated at 37° C. for 30 min, subsequently 1 volume of TE saturated phenol:chloroform is added, stirring for 30 s and centrifuging for 3 min at max. speed in a microcentrifuge, transferring the upper aqueous phase to a new tube. An excess of adapter is then removed as follows:

250 μl of buffer PB are added to the phosphorylation reaction, a QIAQUICK® Spin column is introduced into a 5 ml centrifuge tube, the sample is loaded on the column and centrifuged at 3000 g for 1 min, subsequently washed by adding 0.75 ml of buffer PE and again centrifuged for 1 min, the QIAQUICK® column is transferred to a microcentrifuge tube and centrifuged for 1 min at 10,000 g, thereafter the QIAQUICK® column is put into a 1.5 ml reaction vessel and the DNA is eluted by adding 50 μl 10 m Tris-Cl, pH 8.5, and centrifuging for 1 min at max. speed in a microcentrifuge. Collected eluate: approximately 48 μl.

As the next step, the cDNA is concentrated as follows by ethanol precipitation:

The DNA is mixed with 0.5 volumes of 7.5 M ammonium acetate and 2.5 volumes of cold (−20° C.) 100% ethanol, mixing and placement at −70° C. for 30 min, then it is centrifuged at max. speed in a microcentrifuge for 15 min, and the supernatant solution is removed, the formed pellet is washed with 1 ml of cold (−20° C.) 70% ethanol and centrifuged in a microcentrifuge at max. speed for 5 min, the supernatant solution is removed, the pellet is shortly dried in a vacuum, the sediment is re-suspended in 50 μl of TE buffer for further processing. The DNA concentration is determined by means of UV spectrophotometry at 260 nm.

Then the pCRII vector is subjected to a phosphatase treatment, with the vector being linearized as follows by a restriction cleavage with EcoRI prior to the phosphatase treatment:

Restriction formulation:
1 μg of pCRII DNA
2 μl 10× EcoRI buffer
2 units of EcoRI
Fill up with water to a total volume of 20 μl.

### Incubate for 2 h at 37° C.

Dephosphorylation of vector-DNA:

Addition of 1/10 volume 10× dephosphorylation buffer. Incubation after addition of 1 unit of alkaline phosphatase for 60 min at 37° C. Inactivation of the alkaline phosphatase by heating at 65° C. for 15 min.

Subsequently, the synthesized cDNA is cloned into the EcoRI cleavage site of the vector pCRII.

Ligation formulation:

### 100 ng of Vector-DNA

50 ng of CSF1-cDNA
1 μl of T4 DNA ligase (1 Weiss unit)
1.5 μl of T4 DNA ligase 10× buffer
Fill up with nuclease-free water to 15 μl; the reaction mixture is incubated at room temperature for 3 h, and the pCRII-CSF-1 recombinant plasmid is recovered.

Insertion of the DNA in E. coli:

The plasmid pCRII-CSF-1 is introduced in E. coli by transformation and amplified as follows:

Transformation of bacteria by electroporation
100 μl of electrocompetent E. coli are mixed with half the volume of the ligation formulation (7.5 μl) in cuvettes on ice
electroporation: 25 μF, 2.5 kV, 200Ω
addition of 1 ml of SOC medium for regenerating the cells, transfer of the cells into a tube, and incubation at 37° C. for 1 h, then plating on ampicillin selection plates and growing of the colonies over night at 37° C.

Isolation of the plasmid:

A single colony is picked from the selection plate and incubated in 3 ml of LB with ampicilling for 8 h at 37° C. with vigorous shaking, diluted 1/500 in 100 ml of LB medium, allowed to grow at 37° C. for 12 h under vigorous shaking. Subsequently, the bacteria are harvested by centrifuging at 6000 g for 15 min at 4° C., the bacterial pellets are dissolved in 10 ml of buffer P1, 10 ml of buffer P2 are added, it is thoroughly mixed and incubated for 5 min at room temperature; then 10 ml of ice-cold buffer P3 are added, and it is immediately carefully mixed and incubated on ice for 20 min, centrifuged at 20,000 g for 30 min at 4° C. The supernatant solution is once more centrifuged at 20,000 g for 15 min at 4° C. and transferred to an equilibrated QIAGEN-500 column with 10 ml of buffer QBT. When the column has been washed with 2×30 ml of buffer QC, the DNA is eluted with 15 ml of buffer QF and precipitated to the eluted DNA by adding 10.5 ml of isopropanol (room temperature). After a mixing and an immediate centrifugation at 15,000 g for 30 min at 4° C., the supernatant solution is removed, the DNA pellet is washed with 5 ml 70% ethanol (room temperature), centrifuged at 15,000 g for 10 min, and the supernatant solution is removed. The formed pellet is allowed to air-dry for 5 min, and the DNA is dissolved in 100 μl of TE, pH 8.0. The DNA concentration is determined by means of UV spectrophotometry at 260 nm.

Finally, the sequences of all amplified and cloned CSF-1 constructs are determined by sequencing according to the standard method of Sanger (chain termination method). The CSF-1 constructs may now be used as such or they may be further processed to pharmaceutically acceptable formulations.

### EXAMPLE 2

### Preparation of Gene-Therapeutically Expressible CSF-1 Antisense Constructs

### Preparation of Recombinant Infectious Adenoviruses

The CSF-1 cDNA is excised from plasmid pCRII-CSF-1 of Example 1 and subsequently cloned in antisense orientation into the adenoviral transfer vector:

The insert is excised by restriction cleavage with EcoRI as follows:

Restriction formulation:

1 μg of pCRII-CSF1 DNA
2 μl 10× EcoRI buffer
2 units of EcoRI
Fill up with water to a total volume of 20 μl.

### Incubate at 37° C. for 2 h.

Subsequently, blunt ends are formed in a fill-up reaction with Klenow enzyme with the addition of the following reagents:

2 μl 10×NTB; 1 μl 1 mM dNTP; 1 unit of Klenow, incubate at 37° C. for 15 min and heat at 65° C. for 5-10 min to inactivate the Klenow enzyme.

Thereafter, the transfer vector pQBI-AdCMV5BFP is cleaved with the restriction enzyme BglII:

Restriction formulation:

1 μg of transfer vector DNA

2 μl 10× buffer M

2 units of BglII

It is filled up with water to a total volume of 20 μl and incubated at 37° C. for 2 h, the fragments formed are separated on a 1% TAE agarose gel, the 1641 bp CSF-1 fragment as well as the transfer vector are separately purified from the agarose gel as follows:

Excision of the respective DNA fragment from the agarose gel with a scalpel, weighing of the gel piece, and addition of 3 volumes of the buffer QG to 1 volume of gel, subsequently incubation at 50° C. for 10 min. During the incubation, it is stirred every 2 min, checked whether the color of the mixture is yellow, and subsequently 1 gel volume of isopropanol is added to the sample, mixing. Placing of a QIAQUICK® Spin column in a 2 ml reaction vessel, and application of the sample on the column and centrifuging for 1 min. Put column into a new reaction vessel. Wash by applying 0.75 ml of buffer PE onto the column and centrifuge for 1 min, thereafter removal of the effluent and centrifuging of the column for 1 min at 10,000 g.

Elution of the DNA: Addition of 50 μl of 10 mM Tris-Cl, pH 8.5, and centrifugation for 1 min at max. speed. Subsequently, ligation of the CSF-1 cDNA in the transfer vector pQBI-AdCMV5BFP, namely:

The purified CSF-1 fragment is cloned into the linearized transfer vector as follows:

Ligation formulation:

200 ng of transfer vector DNA
100 ng of CSF-1 cDNA
1 μl of T4 DNA ligase (1 Weiss unit)
1.5 μl of T4 DNA ligase 10× buffer
Fill up with nuclease-free water to 15 μl
Incubate the reaction mixture at room temperature for 6 h.

The subsequent transformation of bacteria by electroporation succeeds as follows:

100 μl of electrocompetent E. coli are mixed with half the volume of the ligation formulation (7.5 μl) in cuvettes under ice-cooling, and electroporation is carried out at 25 μF, 2.5 kV, 200Ω; to regenerate the cells, 1 ml of SOC medium is added, the cells are transferred into a tube and incubated at 37° C. for 1 h, followed by plating on ampicillin selection plates and growing of the colonies over night at 37° C.

Then the plasmid is isolated as follows:

A single colony is taken from the selection plate and incubated in 3 ml of LB with ampicillin for 8 h at 37° C. under vigorous shaking; the starting culture is diluted 1/500 in 100 ml of LB-medium and allowed to grow at 37° C. for 12 h under vigorous shaking; the bacteria are harvested by centrifuging at 6000 g for 15 min at 4° C.; the bacterial pellet is dissolved in 10 ml of buffer P1, 10 ml of buffer P2 are added, it is mixed and incubated at room temperature for 5 min; 10 ml of ice-cold buffer P3 are added and it is carefully mixed immediately and incubated on ice for 20 min, incubated at 20,000 g at 4° C. for 30 min, the supernatant solution is once more centrifuged at 20,000 g and 4° C. for 15 min, and the supernatant solution is transferred to a QIAGEN-500 column that has been equilibrated with 10 ml of buffer QBT, the column is washed with 2×30 ml of buffer Qc, the DNA is eluted with 15 ml of buffer QF and precipitated by adding 10.5 ml of isopropanol (room temperature) to the eluted DNA, mixed, and immediately centrifuged at 15,000 g and 4° C. for 30 min; the supernatant solution is removed. The DNA pellet is washed with 5 ml of 70% ethanol (room temperature) and centrifuged for 10 min at 15,000 g, and the supernatant solution is removed, the pellet is air-dried for 5 min, and the DNA is dissolved in 100 μl of TE, pH 8.0. The DNA concentration is determined by means of UV spectrophotometry at 260 nm. The separation of the fragments is performed on a 1% TAE agarose gel, whereupon the transfer vector is extracted from the agarose gel and purified as follows:

Excision of the linearized vector from the agarose gel with a scalpel, weighing of the gel piece and addition of 3 volumes of buffer QG to 1 volume of gel, incubation at 50° C. for 10 min, wherein it is stirred every 2 min and checked whether the color of the mixture is yellow. Subsequently, addition of 1 gel volume of isopropanol to the sample, mixing, arrangement of a QIAQUICK® Spin column in a 2 ml reaction vessel, and application of the sample on the column and centrifugation for 1 min. Putting column into a new reaction vessel, washing by applying 0.75 ml of buffer PE onto the column and centrifuging for 1 min. Removal of the effluent and centrifuging of the column for 1 min at 10,000 g. Elution of the DNA: Addition of 50 μl of 10 mM Tris-Cl, pH 8.0, and centrifugation at max. speed for 1 min. Yield: approximately 48 μl.

Subsequently, co-transfection of the linearized recombinant transfer vector (pAdCMV5-CSF-BFP) with the viral DNA (AD5CMVlacZE1/E3) in 293 cells is carried out as follows by means of the calcium phosphate method:

Addition of 0.005 volumes of 2 mg/ml carrier DNA to 1×HEBS, mixing by stirring for 1 min. Aliquoting of 2 ml of HEBS plus carrier DNA in a sterile, clear plastics reaction vessel, addition of 20 μg of the linearized recombinant transfer vector (pAdCMV5-CSF-BFP) and 20 μg of the viral DNA to this reaction vessel and careful shaking, subsequently slow addition of 0.1 ml of 2.5 M CaCl₂, careful mixing and incubation at room temperature for 25 min. Addition of 0.5 ml of DNA suspension to a 60 mm cell culture dish with 293 cells, without removal of the growth medium, incubation for 5 h at 37° C. in a CO₂ incubator, removal of the medium and addition of 10 ml of MEMF11-agarose (previously equilibrated at 44° C.). After solidification of the agarose, incubation at 37° C. Plaques appear after 5-14 days. For screening the adenovirus plaque isolate, the plaques are isolated from the transfected culture by cutting out by means of a sterile pasteur pipette and transferred into reaction vessels with 0.5 ml of sterile PBS plus 10% glycerol. Storage at −70° C. until use. Subsequently, removal of the medium from 80% confluent 293 cells in 60 mm dishes, and addition of 0.2 ml of virus (agar suspension). Absorption at room temperature for 30 min. Addition of complete MEMF11+5% Horse serum and incubation at 37° C. Virus harvest and extraction of the infected cellular DNA, when most of the cells have been detached. Careful removal of 4 ml of medium with a pipette, and placement in a reaction vessel with 0.5 ml of sterile glycerol. These virus candidates are stored at −70° C. Removal of the remaining medium from the dish. For DNA extraction from the infected cells, addition of 0.5 ml of pronase solution and incubation at 37° C. for 10 h. Transfer of the lysate into a 1.5 ml reaction vessel, 1× extraction with buffer-saturated phenole, centrifuging for 10 min and collection of the upper, aqueous phase and transfer into a new vessel, addition of 1 ml of ethanol for precipitating the DNA. Thorough mixing. Centrifuging for 10 min at 14,000 rpm, sucking off of the supernatant solution, washing of the pellet with 70% ethanol, centrifuging for 5 min, sucking off of the supernatant, and air-drying of the pellet. Dissolving of the DNA in 50 μl of 0.1×SSC and carrying out a restriction cleavage with 5 μl with Hind III (1 unit over night) Application of the digested sample onto a 1% agarose gel with ethidium bromide. Viral DNA bands then are easily visible under UV light, via a background smear of cellular DNA. Verification of recombinant virus candidates by further diagnostic restriction enzyme cleavages, as well as by checking the expression of the BFP (blue fluorescent protein) under the fluorescence microscope. Correct recombinants are further purified by 2 further rounds by means of plaque purification and screened before a high titer stock is prepared.

Subsequently, plaque assays are carried out for a purification and titration of recombinant adenoviruses (AD5CMV-CSF) as follows:

Sucking off the medium from confluent 293 cells in 60 mm dishes. Addition of 0.2 ml of virus (10⁻³-10⁻⁶ dilutions of an agar suspension in PBS for plaque purification or 10⁻³-10⁻⁹ dilutions of the stock solution for titration). Absorption of the virus for 40 min at room temperature. Addition of 10 ml of MEMF11 agarose overlay, cooling and incubation at 37° C. Plaques are counted for titration after 7 and after 10 days. Plaque purification: isolation of the plaques as described above.

Finally, high-titer viral stock solutions of monolayer cells are prepared as follows:

Ten 150 mm dishes are plated with 293 cells, and after having reached a confluence of 80%, they are infected; subsequently, for preparing a high-titer stock, the medium is removed from the 293 cells and infection is carried out with a multiplicity of infection (MOI) of 1-10 PFU per cell (1 ml of virus suspension per 150 mm dish), it is absorbed for 40 min, followed by the addition of MEMF11+5% horse serum, incubation at 37° C. and daily check for signs of the cytopathic effect. When the cytopathic effect is almost complete, harvest by scraping the cells off the dish, combination of the cells plus medium and centrifugation at 800 g for 15 min. Sucking off the medium and resuspending the cell pellet in 2 ml of PBS+10% glycerol per 150 mm dish. Further purification of the virus solution by CsCl density gradient centrifugation, followed by dialysis against 10 mM Tris-HCl, pH 8.0. Addition of sterile glycerol to a final concentration of 10% and storage at −70° C.

Thus, recombinant infectious high-titer adenoviral CSF-1 antisense constructs are recovered which may be used directly for a gene therapy.

### EXAMPLE 3

### Use of the Recombinant Infectious Adenoviral CSF-1 Antisense Constructs in the Infection of Cells

Tested cell systems:

Lewis lung carcinoma cells,
colon carcinoma cells,
mamma carcinoma cells,
germinal tumor cells.

Carrying out the infection:

To cell monolayers in 60 mm dishes with 80% confluence, 0.5 ml virus solution (AD5CMV-CSF) of different MOIs (multiplicities of infection) are added and incubated for 30 min at room temperature.

The control infections are carried out with: AD5CMVlacZ E1/E3 (adenovirus without CSF-insert), AdenoGFP (adenovirus with GFP), mock infection (culture medium). Addition of 6 ml of medium and incubation of the dishes in a CO₂ incubator (37° C., 5% CO₂).

The infection showed the following effects:

CSF-1 gene expression in the host cells was markedly lowered (reduction of the mRNA-(CSF-1 protein)-level in all the transfected cells to <30% as compared to non-treated wild type cells.

New vessel formation inhibited, anti-oncogenic effects (retarded cell growth).

When analysing the cell cycle, the triggering of apoptosis was found.

In viral transfection (adenoviruses) of mice, an intra-tumoral administration of 1×10⁹−5×10¹⁰ PFU in a volume of 0.5 ml at the most was carried out.

An intratracheal administration was carried out by administering up to 2×10⁹ PFU of the recombinant virus in a volume of from 23-35 μl, wherein, in particular in the intra-tumoral administration, a nearly complete retrogression of the tumors could be observed.

### EXAMPLE 4

### Preparation of CSF-1 Specific Oligonucleotides and Use Thereof

### Synthesis of a CSF-1-Specific Oligonucleotide

The synthesis of the CSF-1-specific oligonucleotide 5′-GCCCGGCGCGGTCA-3′ (SEQ ID NO: 8)(14-mer, homologous to the first 14 nucleotides of the CSF-1-cDNA primary sequence following the start codon (ATG) of base pair 120-106) is carried out on an automated oligonucleotide synthesizer which is based on the phosphorous-amidite-method. The synthesis is carried out in the 3′-5′ orientation of the given nucleotide sequence, and when the synthesis has been finished, the column with the bound oligonucleotide at first is washed with 3 ml of concentrated NH₃. The procedure is repeated several times so that the column is completely permeated with NH₃. The column is incubated with NH₃ for approximately 2 h at RT (rinsed several times), and finally the oligonucleotide is recovered. In a tightly closed reaction vessel, the solution is heated at 55° C. for 16 h. In a rotary evaporator, the NH₃ is removed (30 μl of triethylamine are added to prevent detritylation).

### Preparation of Phosphorothioate-Oligonucleotides with the Beaucage Reagent

Sulphuration is carried out with 240 μl of a 0.05 M solution of 3H-1,2-benzodithiol-3-one-1,1-dioxide (Pharmacia, Sigma) directly on the column, even before the capping step, for which purpose an appropriate reaction flask is filled with a mixture of 12 ml of dichlorodimethylsilane and 200 ml of dichloromethane, whereupon the solution is removed after 5 min and the flask is rinsed with methanol. Subsequently, the flask is dried over night at 110° C. and cooled in an exsiccator. 0.5 g (2.5 mmol) of the Beaucage reagent are dissolved in 50 ml of dry acetonitrile and filled into the reaction flask. The reaction flask is connected to the oligosynthesizer, and the reagent is pumped over the column (2×).

Analytical Reverse-Phase HPLC

(column: silica-bound C-18 phase with spherical particles (5 μm, 300 A pore size), additionally nucleosil 100-5 C-18, 100 mm×4 mm).

The unpurified oligonucleotide (evaporated sediment) is taken up in 300 μl 0.1 M triethylammonium acetate buffer, pH 7.

An UV monitor is set at 260 nm, and the flow rate is adjusted to 1 ml/min. A buffer gradient of 0-50% 1 of an 0.1 M triethylammonium acetate buffer, pH 7, in 80% acetonitrile is run for 50 min. Subsequently, the same buffer is increased from 50-100% within 5 min. A small sample portion of 5 μl of the unpurified oligonucleotide (2-5% of the total amount) is applied to the column, the absorption is recorded at 260 nm.

Preparative Reverse-Phase HPLC

The same arrangement as in the synthesis of a CSF-1-specific oligonucleotide is used, except that the total amount of 300 μl of the oligonucleotide solution is applied to the column, and the absorption at 260 nm is followed, and the central parts of the eluted peaks are collected, the combined samples are sedimented in a rotary evaporator, 1 ml of 80% acetic acid is added to the dried samples and incubated for 1 h at RT, and the samples are again sedimented in the rotary evaporator.

The pellet is dissolved in 1 ml of dist./ster. H₂O, twice extracted with DMT-OH and once with ethyl acetat. The samples are dried and sedimented in the rotary evaporator. The precipitate is dissolved in a certain volume of dist./ster. H₂O, and the extinction is measured at 260 nm so as to determine the amount (dilution 1:100, taking into consideration the sequence-dependent extinction coefficient).

Use of CSF-1 Antisense Phosphorothioate Oligonucleotides

The CSF-1 antisense phosphorothioate-modified oligonucleotides prepared are applied in various ways as a water-soluble pure substance (HPLC-purified) and dissolved in PBS. What was examined were the systemic administration by means of intravenous injection, the intraarterial administration, wherein the supplying organ-specific vessel was considered as the artery of choice so as to allow for an administration of the substance as close to the target as possible. In dependence on the localization of the tumor, other routes, e.g. topical or intraperitoneal administration were examined. Furthermore, osmotic mini pumps can serve as reservoirs (primarily with mice as test animals) in which the CSF1 antisense is filled and administered by subcutaneous or intravenous implantation. The advantage of this method resides in the simple and reliable mode of application. Moreover, the pumps have the advantage that, once implanted, they guarantee the application of constant rates for up to 4 weeks. In this case, the dose of the CSF-1 antisense oligonucleotides to be administered is within the milligram range. Thus, in an anti-oncogenic therapy on mice, doses of 0.1-20 mg/kg body weight/day were used. Rats having a body weight of g each receive 100 μl doses intravenously at a concentration of 0.1-1 μg/ml. In the human system, a dosage of 0.05 mg/kg/h is appropriate, since at this dosage toxic effects do not yet occur due to the administration of modified oligonucleotides alone. CSF-1 antisense oligonucleotide treatment regimens should be maintained continuously for at least 2 weeks.

1. The use of inhibitors of CSF-1 activity for preparing a medicament for the treatment of tumor diseases.

2. The use according to claim 1, characterised in that a neutralizing antibody against CSF-1 or its receptor is employed as the inhibitor of CSF-1 activity.

3. The use according to claim 1, characterised in that an antisense nucleic acid against CSF-1 or its receptor is employed as the inhibitor of CSF-1 activity.

4. The use according to claim 1, characterised in that the medicament is prepared to inhibit the growth of solid tumors.

5. The use according to claim 1, characterised in that the medicament is prepared to retard malignant tumor diseases.

6. The use according to claim 1, characterised in that a genetically altered cell is used as the inhibitor of CSF-1 activity, in which cell the activity of CSF-1 or its receptor is inhibited by the genetic alteration, in particular by deletion of at least parts of the gene for CSF-1 or its receptor.

7. The use according to claim 1, characterised in that the medicament is formulated for intra-tumoral administration.

8. The use according to claim 1, characterised in that the medicament is prepared for the treatment of solid tumors selected from the group of germinal tumors, epithelial tumors and adenocarcinomas.
